(19) United States
(12) Patent Application Publication
(10) Pub. No.: US 2016/0375237 A1
(43) Pub. Date: Dec. 29, 2016

(54) SYSTEMS AND METHODS FOR MAKING AND USING ELECTRICAL STIMULATION LEADS WITH COATED CONTACTS

(71) Applicant: Boston Scientific Neuromodulation Corporation, Valencia, CA (US)

(72) Inventors: Benjamin Phillip Hahn, Stevenson Ranch, CA (US); Joshua Dale Howard, Sacramento, CA (US)

(21) Appl. No.: 15/188,844

(22) Filed: Jun. 21, 2016

Related U.S. Application Data
(60) Provisional application No. 62/183,665, filed on Jun. 23, 2015.

(51) Int. Cl. A61N 1/05; A61N 1/375

(52) U.S. Cl. CPC A61N 1/05; A61N 1/3752

(57) ABSTRACT

An electrical stimulation lead includes a lead body having a distal end portion, a proximal end portion, and a longitudinal length; electrodes disposed along the distal end portion of the lead body; terminals disposed along the proximal end portion of the lead body; and conductors electrically coupling the plurality of terminals to the plurality of electrodes. At least one of the electrodes or terminals is a coated contact. Each coated contact includes a conductive substrate and a conductive coating disposed on the substrate.

### CROSS-REFERENCE TO RELATED APPLICATIONS

This application claims the benefit under 35 U.S.C. §119(e) of U.S. Provisional Patent Application Ser. No. 62/183,665, filed Jun. 23, 2015, which is incorporated herein by reference.

### FIELD

The present invention is directed to the area of implantable electrical stimulation systems and methods of making and using the systems. The present invention is also directed to implantable electrical stimulation leads with coated contacts (electrodes or terminals), as well as methods of making and using the leads and electrical stimulation systems having the leads.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat incontinence, as well as a number of other applications under investigation. Functional electrical stimulation systems have been applied to restore some functionality to paralyzed extremities in spinal cord injury patients. Stimulation of the brain, such as deep brain stimulation, can be used to treat a variety of diseases or disorders.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

One embodiment is an electrical stimulation lead including a lead body having a distal end portion, a proximal end portion, and a longitudinal length; electrodes disposed along the distal end portion of the lead body; terminals disposed along the proximal end portion of the lead body; and conductors electrically coupling the plurality of terminals to the plurality of electrodes. At least one of the electrodes or terminals is a coated contact. Each coated contact includes a conductive substrate and a conductive coating disposed on the substrate.

In at least some embodiments, each of the electrodes (or each of the terminals) is a one of the coated contacts. In at least some embodiments, the conductive substrate and the conductive coating are different materials. In at least some embodiments, the conductive coating is a conductive metal or a conductive metal oxide or a conductive metal nitride or a conductive polymer.

In at least some embodiments, the substrate of the coated contact includes an opening receiving a portion of a one of the conductors and a fastening portion engaging the portion of the conductor to fasten the portion to the coated contact. In at least some embodiments, the substrate of the coated contact includes at least two extensions that define a gap receiving a portion of a one of the conductors with the at least two extensions engaging the portion of the conductor to fasten the portion to the coated contact. In at least some embodiments, the substrate of the coated contact includes a tab extending away from a remainder of the substrate and to which one of the conductors is attached. In at least some embodiments, the tab is not radially beneath the conductive coating. In at least some embodiments, the tab is thinner than a remainder of the substrate.

In at least some embodiments, the coated contact is a ring contact. In at least some embodiments, the coated contact extends no more than 50% around a circumference of the lead. In at least some embodiments, the conductive coating is coated on the substrate using physical vapor deposition or chemical vapor deposition. In at least some embodiments, the conductive coating is coated on the substrate using electrodeposition or dip coating.

Another embodiment is an electrical stimulating system including any of the electrical stimulation leads described above; a control module coupleable to the electrical stimulation lead, the control module including a housing, and an electronic subassembly disposed in the housing; and a connector for receiving the electrical stimulation lead, the connector having a proximal end, a distal end, and a longitudinal length, the connector including a connector housing defining a port at the distal end of the connector, the port configured and arranged for receiving the proximal end of the lead body of the electrical stimulation lead, and connector contacts disposed in the connector housing and configured and arranged to couple to at least one of the terminals disposed on the proximal end of the lead body of the electrical stimulation lead.

In at least some embodiments, the electrical stimulation system further includes a lead extension coupleable to both the electrical stimulation lead and the control module.

A further embodiment is a method of making any of the electrical stimulation leads described above. The method includes disposing the electrodes on a non-conductive mount, wherein at least one of the electrodes is a one of the coated contacts; attaching the conductors to the electrodes; and molding a lead body around the electrodes, the conductors, and the mount.

In at least some embodiments, the substrate of the coated contact includes an opening and a fastening portion disposed around the opening, where the step of attaching the conductors to the electrodes includes inserting a portion of a one of the conductors into the opening and crimping the fastening portion to fasten the portion to the coated contact. In at least some embodiments, the substrate of the coated contact includes at least two extensions that define a gap, where the step of attaching the conductors to the electrodes includes inserting a portion of a one of the conductors into the gap and crimping the at least two extensions to fasten the portion to the coated contact. In at least some embodiments, the substrate of the coated contact includes a tab extending away from, and not radially beneath, the conductive coating, where the step of attaching the conductors to the electrodes includes welding a one of the conductors to the tab.

In at least some embodiments, the method further includes forming the coated contact by coating the substrate with the conductive coating using physical vapor deposition or chemical vapor deposition. In at least some embodiments, the method further includes forming the coated contact by coating the substrate with the conductive coating using electrodeposition or dip coating. In at least some embodiments, the method includes forming the coated contact by coating the substrate with multiple conductive layers.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an implantable medical device that includes a paddle body coupled to a control module via lead bodies, according to the invention;

FIG. 2 is a schematic view of another embodiment of an implantable medical device that includes a percutaneous lead body coupled to a control module via a lead body, according to the invention;

FIG. 3A is a schematic view of one embodiment of a plurality of connectors disposed in the control module of FIG. 1, the connectors configured and arranged to receive the proximal portions of the lead bodies of FIG. 1, according to the invention;

FIG. 3B is a schematic view of one embodiment of a connector disposed in the control module of FIG. 2, the connector configured and arranged to receive the proximal portion of one of the lead body of FIG. 2, according to the invention;

FIG. 3C is a schematic view of one embodiment of a proximal portion of the lead body of FIG. 2, a lead extension, and the control module of FIG. 2, the lead extension configured and arranged to couple the lead body to the control module, according to the invention;

FIG. 4 is a schematic side view of yet another embodiment of an implantable medical device for brain stimulation, according to the invention;

FIG. 5A is a schematic perspective view of one embodiment of a distal end of a lead containing segmented electrodes, according to the invention;

FIG. 5B is a schematic perspective view of a second embodiment of a distal end of a lead containing segmented electrodes, according to the invention;

FIG. 5C is a schematic perspective view of a third embodiment of a distal end of a lead containing segmented electrodes, according to the invention;

FIG. 5D is a schematic perspective view of a fourth embodiment of a distal end of a lead containing segmented electrodes, according to the invention;

FIG. 5E is a schematic perspective view of a fifth embodiment of a distal end of a lead containing segmented electrodes, according to the invention;

FIG. 5F is a schematic perspective view of a sixth embodiment of a distal end of a lead containing segmented electrodes, according to the invention;

FIG. 5G is a schematic perspective view of a seventh embodiment of a distal end of a lead containing segmented electrodes, according to the invention;

FIG. 6 is a schematic transverse cross-sectional view of one embodiment of a coated contact, according to the invention;

FIG. 7 is a schematic transverse cross-sectional view of a second embodiment of a coated contact, according to the invention;

FIG. 8A is a schematic top view of a third embodiment of a coated contact, according to the invention;

FIG. 8B is a schematic transverse cross-sectional view of the coated contact of

FIG. 8A, according to the invention;

FIG. 9A is a schematic top view of a third embodiment of a coated segmented contact, according to the invention;

FIG. 9B is a schematic transverse cross-sectional view of the coated segmented contact of FIG. 9A, according to the invention; and

FIG. 10 is a schematic overview of one embodiment of components of an electrical stimulation system, according to the invention.

### DETAILED DESCRIPTION

The present invention is directed to the area of implantable electrical stimulation systems and methods of making and using the systems. The present invention is also directed to implantable electrical stimulation leads with coated contacts (electrodes or terminals), as well as methods of making and using the leads and electrical stimulation systems having the leads.

Suitable implantable electrical stimulation systems include, but are not limited to, an electrode lead (“lead”) with one or more electrodes disposed on a distal end of the lead and one or more terminals disposed on one or more proximal ends of the lead. Leads include, for example, deep brain stimulation leads, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734;7,761,165; 7,783,359; 7,792,590; 7,809,446; 7,949,395; 7,974,706; 6,175,710; 6,224,450; 6,271,094; 6,295,944; 6,364,278; 6,391,985; 8,831,742; and 8,688,235; U.S. Patent Applications Publication Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; and 2013/0105071, all of which are incorporated by reference.

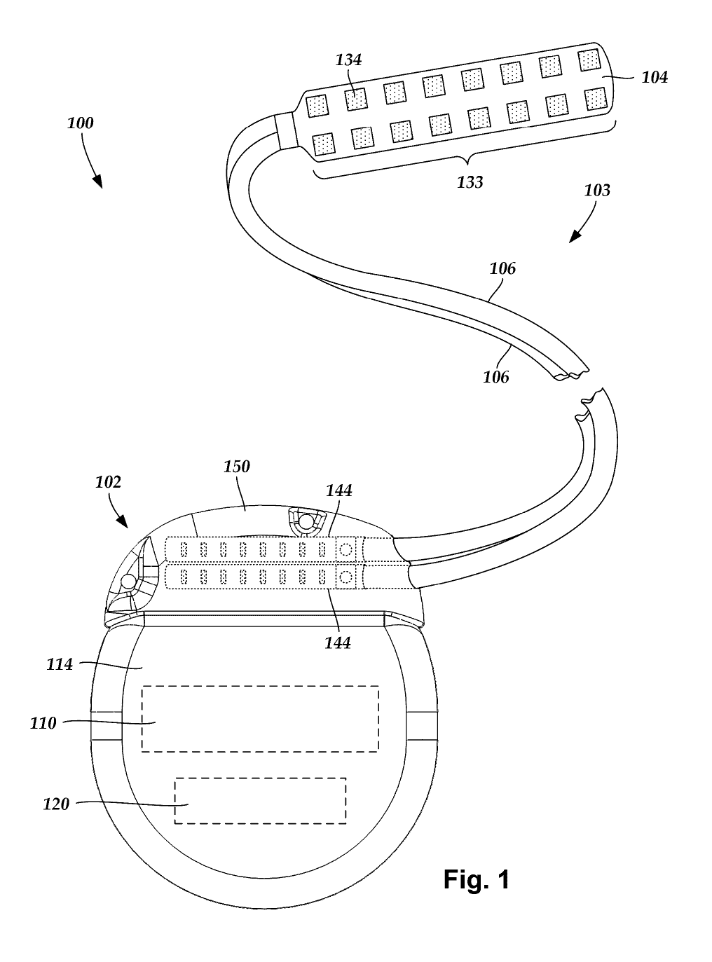

FIG. 1 illustrates schematically one embodiment of an electrical stimulation system 100. The electrical stimulation system includes a control module (e.g., a stimulator or pulse generator) 102 and a lead 103. The lead 103 including a paddle body 104 and one or more lead bodies 106 coupling the control module 102 to the paddle body 104. The paddle body 104 and the one or more lead bodies 106 form the lead 103. The paddle body 104 typically includes a plurality of electrodes 134 that form an array of electrodes 133. The control module 102 typically includes an electronic subassembly 110 and an optional power source 120 disposed in a sealed housing 114. In FIG. 1, two lead bodies 106 are shown coupled to the control module 102.

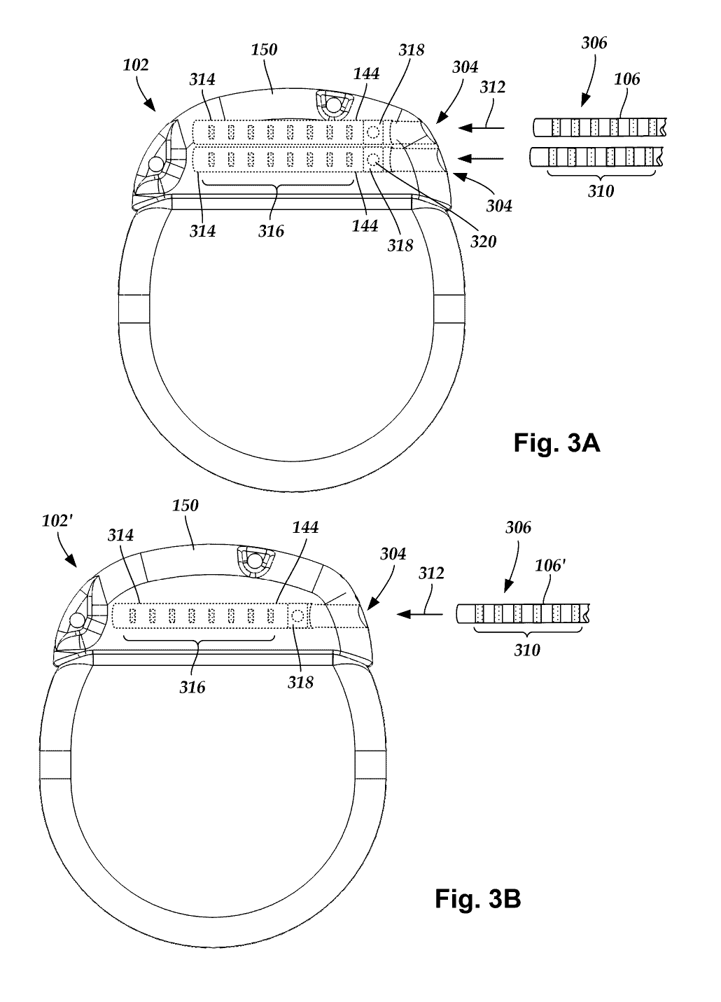

The control module 102 typically includes one or more connectors 144 into which the proximal end of the one or more lead bodies 106 can be plugged to make an electrical connection via connector contacts (e.g., 316 in FIG. 3A) disposed in the connector 144 and terminals (e.g., 310 in FIG. 3A) on each of the one or more lead bodies 106. The connector contacts are coupled to the electronic subassembly 110 and the terminals are coupled to the electrodes 134. In FIG. 1, two connectors 144 are shown.

The one or more connectors 144 may be disposed in a header 150. The header 150 provides a protective covering over the one or more connectors 144. The header 150 may be formed using any suitable process including, for example, casting, molding (including injection molding), and the like. In addition, one or more lead extensions 324 (see FIG. 3C) can be disposed between the one or more lead bodies 106 and the control module 102 to extend the distance between the one or more lead bodies 106 and the control module 102.

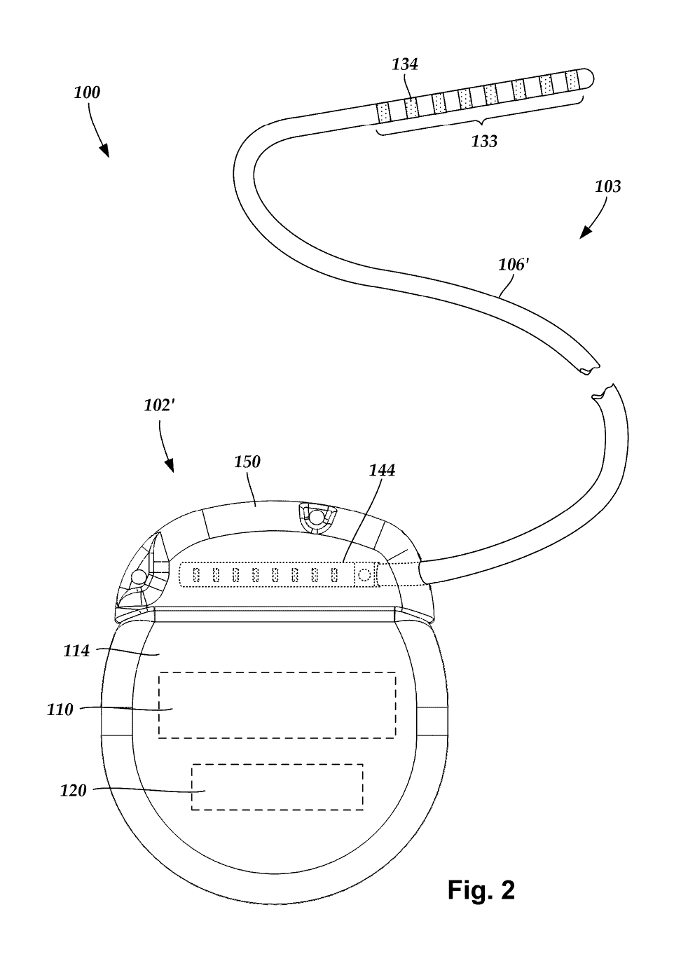

It will be understood that the electrical stimulation system can include more, fewer, or different components and can have a variety of different configurations including those configurations disclosed in the electrical stimulation system references cited herein. For example, instead of a paddle body 104, the electrodes 134 can be disposed in an array at or near the distal end of a lead body 106′ forming a percutaneous lead 103, as illustrated in FIG. 2. The percutaneous lead may be isodiametric along the length of the lead body 106″. The lead body 106′ can be coupled with a control module 102′ with a single connector 144.

The electrical stimulation system or components of the electrical stimulation system, including one or more of the lead bodies 106, the control module 102, and, in the case of a paddle lead, the paddle body 104, are typically implanted into the body of a patient. The electrical stimulation system can be used for a variety of applications including, but not limited to, spinal cord stimulation, brain stimulation, neural stimulation, muscle activation via stimulation of nerves innervating muscle, and the like.

The number of electrodes 134 in the array of electrodes 133 may vary. For example, there can be two, three, four, five, six, seven, eight, nine, ten, eleven, twelve, thirteen, fourteen, fifteen, sixteen, or more electrodes 134. As will be recognized, other numbers of electrodes 134 may also be used. In FIG. 1, sixteen electrodes 134 are shown. The electrodes 134 can be formed in any suitable shape including, for example, round, oval, triangular, rectangular, pentagonal, hexagonal, heptagonal, octagonal, or the like.

The electrodes of the paddle body 104 or one or more lead bodies 106 are typically disposed in, or separated by, a non-conductive, biocompatible material including, for example, silicone, polyurethane, and the like or combinations thereof.

The paddle body 104 and one or more lead bodies 106 may be formed in the desired shape by any process including, for example, molding (including injection molding), casting, and the like. Electrodes and connecting wires can be disposed onto or within a paddle body either prior to or subsequent to a molding or casting process. The non-conductive material typically extends from the distal end of the lead 103 to the proximal end of each of the one or more lead bodies 106. The non-conductive, biocompatible material of the paddle body 104 and the one or more lead bodies 106 may be the same or different. The paddle body 104 and the one or more lead bodies 106 may be a unitary structure or can be formed as two separate structures that are permanently or detachably coupled together.

Terminals (e.g., 310 in FIG. 3A) are typically disposed at the proximal end of the one or more lead bodies 106 for connection to corresponding conductive contacts (e.g., 316 in FIG. 3A) in connectors (e.g., 144 in FIG. 1) disposed on, for example, the control module 102 (or to other devices, such as conductive contacts on a lead extension, an operating room cable, a splitter, an adaptor, or the like).

Conductive wires (not shown) extend from the terminals (e.g., 310 in FIG. 3A) to the electrodes 134. Typically, one or more electrodes 134 are electrically coupled to a terminal (e.g., 310 in FIG. 3A). In some embodiments, each terminal (e.g., 310 in FIG. 3A) is only coupled to one electrode 134.

The conductive wires may be embedded in the non-conductive material of the lead or can be disposed in one or more lumens (not shown) extending along the lead. In some embodiments, there is an individual lumen for each conductive wire. In other embodiments, two or more conductive wires may extend through a lumen. There may also be one or more lumens (not shown) that open at, or near, the proximal end of the lead, for example, for inserting a stylet rod to facilitate placement of the lead within a body of a patient. Additionally, there may also be one or more lumens (not shown) that open at, or near, the distal end of the lead, for example, for infusion of drugs or medication into the site of implantation of the paddle body 104. The one or more lumens may, optionally, be flushed continually, or on a regular basis, with saline, epidural fluid, or the like. The one or more lumens can be permanently or removably sealable at the distal end.

As discussed above, the one or more lead bodies 106 may be coupled to the one or more connectors 144 disposed on the control module 102. The control module 102 can include any suitable number of connectors 144 including, for example, two three, four, five, six, seven, eight, or more connectors 144. It will be understood that other numbers of connectors 144 may be used instead. In FIG. 1, each of the two lead bodies 106 includes eight terminals that are shown coupled with eight conductive contacts disposed in a different one of two different connectors 144.

FIG. 3A is a schematic side view of one embodiment of a plurality of connectors 144 disposed on the control module 102. In at least some embodiments, the control module 102 includes two connectors 144. In at least some embodiments, the control module 102 includes four connectors 144. In FIG. 3A, proximal ends 306 of the plurality of lead bodies 106 are shown configured and arranged for insertion to the control module 102. FIG. 3B is a schematic side view of one embodiment of a single connector 144 disposed on the control module 102′. In FIG. 3B, the proximal end 306 of the single lead body 106′ is shown configured and arranged for insertion to the control module 102′.

In FIGS. 3A and 3B, the one or more connectors 144 are disposed in the header 150. In at least some embodiments, the header 150 defines one or more lumens 304 into which the proximal end(s) 306 of the one or more lead bodies 106/106′ with terminals 310 can be inserted, as shown by directional arrows 312, in order to gain access to the connector contacts disposed in the one or more connectors 144.

The one or more connectors 144 each include a connector housing 314 and a plurality of connector contacts 316 disposed therein. Typically, the connector housing 314 provides access to the plurality of connector contacts 316 via the lumen 304. In at least some embodiments, one or more of the connectors 144 further includes a retaining element 318 configured and arranged to fasten the corresponding lead body 106/106′ to the connector 144 when the lead body 106/106′ is inserted into the connector 144 to prevent undesired detachment of the lead body 106/106′ from the connector 144. For example, the retaining element 318 may include an aperture 320 through which a fastener (e.g., a set screw, pin, or the like) may be inserted and secured against an inserted lead body 106/106′.

When the one or more lead bodies 106/106′ are inserted into the one or more lumens 304, the connector contacts 316 can be aligned with the terminals 310 disposed on the one or more lead bodies 106/106′ to electrically couple the control module 102 to the electrodes (134 of FIG. 1) disposed at a distal end of the one or more lead bodies 106. Examples of connectors in control modules are found in, for example, U.S. Pat. Nos. 7,244,150 and 6,224,450, which are incorporated by reference.

In at least some embodiments, the electrical stimulation system includes one or more lead extensions. The one or more lead bodies 106/106′ can be coupled to one or more lead extensions which, in turn, are coupled to the control module 102/102′. In FIG. 3C, a lead extension connector 322 is disposed on a lead extension 324. The lead extension connector 322 is shown disposed at a distal end 326 of the lead extension 324. The lead extension connector 322 includes a connector housing 344. The connector housing 344 defines at least one lumen 330 into which a proximal end 306 of the lead body 106′ with terminals 310 can be inserted, as shown by directional arrow 338. The lead extension connector 322 also includes a plurality of connector contacts 340. When the lead body 106′ is inserted into the lumen 330, the connector contacts 340 disposed in the connector housing 344 can be aligned with the terminals 310 on the lead body 106 to electrically couple the lead extension 324 to the electrodes (134 of FIG. 1) disposed at a distal end (not shown) of the lead body 106′.

The proximal end of a lead extension can be similarly configured and arranged as a proximal end of a lead body. The lead extension 324 may include a plurality of conductive wires (not shown) that electrically couple the connector contacts 340 to terminal on a proximal end 348 of the lead extension 324. The conductive wires disposed in the lead extension 324 can be electrically coupled to a plurality of terminals (not shown) disposed on the proximal end 348 of the lead extension 324. In at least some embodiments, the proximal end 348 of the lead extension 324 is configured and arranged for insertion into a lead extension connector disposed in another lead extension. In other embodiments (as shown in FIG. 3C), the proximal end 348 of the lead extension 324 is configured and arranged for insertion into the connector 144 disposed on the control module 102′.

It will be understood that the control modules 102/102′ can receive either lead bodies 106/106′ or lead extensions 324. It will also be understood that the electrical stimulation system 100 can include a plurality of lead extensions 324. For example, each of the lead bodies 106 shown in FIGS. 1 and 3A can, alternatively, be coupled to a different lead extension 324 which, in turn, are each coupled to different ports of a two-port control module, such as the control module 102 of FIGS. 1 and 3A.

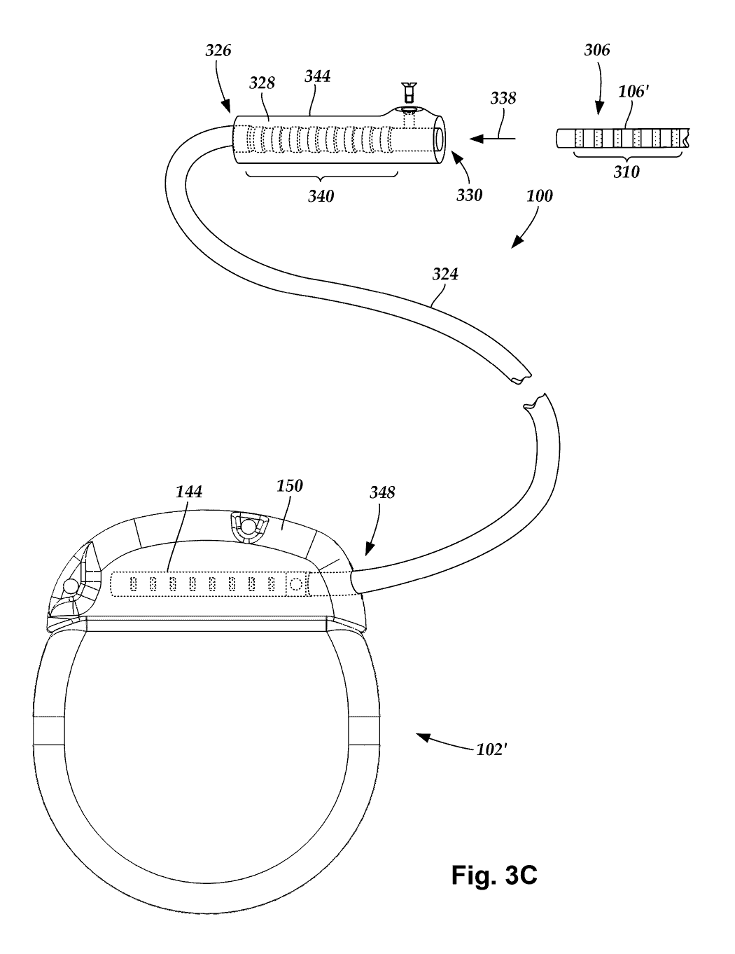

Turning to FIG. 4, in the case of deep brain stimulation, the lead may include stimulation electrodes, recording electrodes, or a combination of both. At least some of the stimulation electrodes, recording electrodes, or both are provided in the form of segmented electrodes that extend only partially around the perimeter (for example, the circumference) of the lead. These segmented electrodes can be provided in sets of electrodes, with each set having electrodes circumferentially distributed about the lead at a particular longitudinal position.

In at least some embodiments, a practitioner may determine the position of the target neurons using recording electrode(s) and then position the stimulation electrode(s) accordingly. In some embodiments, the same electrodes can be used for both recording and stimulation. In some embodiments, separate leads can be used; one with recording electrodes which identify target neurons, and a second lead with stimulation electrodes that replaces the first after target neuron identification. In some embodiments, the same lead may include both recording electrodes and stimulation electrodes or electrodes may be used for both recording and stimulation.

FIG. 4 illustrates one embodiment of a device 400 for brain stimulation. The device includes a lead 410, a plurality of electrodes 425 disposed at least partially about a perimeter of the lead 410, a plurality of terminals 435, a connector 444 for connection of the electrodes to a control unit, and a stylet 440 for assisting in insertion and positioning of the lead in the patient's brain. The stylet 440 can be made of a rigid material. Examples of suitable materials for the stylet include, but are not limited to, tungsten, stainless steel, and plastic. The stylet 440 may have a handle 450 to assist insertion into the lead 410, as well as rotation of the stylet 440 and lead 410. The connector 444 fits over a proximal end of the lead 410, preferably after removal of the stylet 440.

In FIG. 4, the electrodes 425 are shown as including both ring electrodes, such as ring electrode 420, and segmented electrodes, such as segmented electrodes 430. In some embodiments, the electrodes 425 are all segmented. In other embodiments, the electrodes 425 are all ring-shaped. In FIG. 4, each of the terminals 435 is shown as being ring-shaped. The segmented electrodes of FIG. 4 are shown in sets of two, where the two segmented electrodes of a particular set are electrically isolated from one another and are circumferentially-offset along the lead 410. Any suitable number of segmented electrodes can be formed into a set including, for example, two, three, four, or more segmented electrodes.

Segmented electrodes can be used to direct stimulus current to one side, or even a portion of one side, of the lead. When segmented electrodes are used in conjunction with an implantable pulse generator that delivers current stimulus, current steering can be achieved to more precisely deliver the stimulus to a position around an axis of the lead (i.e., radial positioning around the axis of the lead). Segmented electrodes may provide for superior current steering than ring electrodes because target structures in deep brain stimulation are not typically symmetric about the axis of the distal electrode array. Instead, a target may be located on one side of a plane running through the axis of the lead. Through the use of a segmented electrode array, current steering can be performed not only along a length of the lead but also around a perimeter of the lead. This provides precise three-dimensional targeting and delivery of the current stimulus to neural target tissue, while potentially avoiding stimulation of other tissue. Examples of leads with segmented electrodes include U.S. Pat. Nos. 6,295,944; and 6,391,985; and U.S. Patent Applications Publication Nos. 2011/0005069; 2010/0268298; 2011/0130817; 2011/0130818; 2011/0078900; 2011/0238129; 2011/0313500; 2012/0016378; 2012/0046710; 2012/0165911; 2012/0197375; 2012/0203316; 2012/0203320; and 2012/0203321, all of which are incorporated herein by reference.

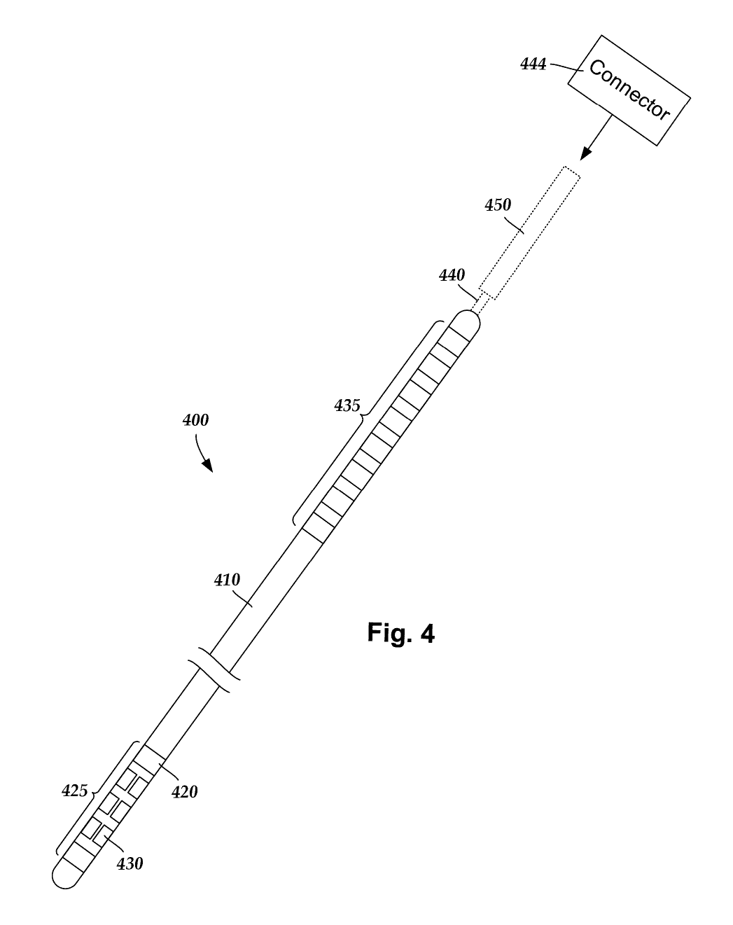

FIGS. 5A-5H illustrate leads 500 with segmented electrodes 550, optional ring electrodes 520 or tip electrodes 520a, and a lead body 510. The sets of segmented electrodes 550 each include either two (FIG. 5B), three (FIGS. 5E-5H), or four (FIGS. 5A, 5C, and 5D) or any other number of segmented electrodes including, for example, three, five, six, or more. The sets of segmented electrodes 550 can be aligned with each other (FIGS. 5A-5G) or staggered (FIG. 5H).

When the lead 500 includes both ring electrodes 520 and segmented electrodes 550, the ring electrodes 520 and the segmented electrodes 550 may be arranged in any suitable configuration. For example, when the lead 500 includes two ring electrodes 520 and two sets of segmented electrodes 550, the ring electrodes 520 can flank the two sets of segmented electrodes 550 (see e.g., FIGS. 1, 5A, and 5E-5H). Alternately, the two sets of ring electrodes 520 can be disposed proximal to the two sets of segmented electrodes 550 (see e.g., FIG. 5C), or the two sets of ring electrodes 520 can be disposed distal to the two sets of segmented electrodes 550 (see e.g., FIG. 5D). One of the ring electrodes can be a tip electrode (see, tip electrode 520a of FIGS. 5E and 5G). It will be understood that other configurations are possible as well (e.g., alternating ring and segmented electrodes, or the like).

By varying the location of the segmented electrodes 550, different coverage of the target neurons may be selected. For example, the electrode arrangement of FIG. 5C may be useful if the physician anticipates that the neural target will be closer to a distal tip of the lead body 510, while the electrode arrangement of FIG. 5D may be useful if the physician anticipates that the neural target will be closer to a proximal end of the lead body 510.

Any combination of ring electrodes 520 and segmented electrodes 550 may be disposed on the lead 500. For example, the lead may include a first ring electrode 520, two sets of segmented electrodes; each set formed of four segmented electrodes 550, and a final ring electrode 520 at the end of the lead. This configuration may simply be referred to as a 1-4-4-1 configuration (FIGS. 5A and 5E—ring electrodes 520 and segmented electrode 550). It may be useful to refer to the electrodes with this shorthand notation. Thus, the embodiment of FIG. 5C may be referred to as a 1-1-1-4 configuration, while the embodiment of FIG. 5D may be referred to as a 1-4-1-1 configuration. The embodiments of FIGS. 5F, 5G, and 5H can be referred to as a 1-3-3-1 configuration. Other electrode configurations include, for example, a 2-2-2-2 configuration, where four sets of segmented electrodes are disposed on the lead, and a 1-4 configuration, where two sets of segmented electrodes, each having four segmented electrodes 550 are disposed on the lead. The 1-3-3-1 electrode configuration of FIGS. 5F, 5G, and 5H has two sets of segmented electrodes, each set containing three electrodes disposed around the perimeter of the lead, flanked by two ring electrodes (FIGS. 5F and 5H) or a ring electrode and a tip electrode (FIG. 5G). In some embodiments, the lead includes 16 electrodes. Possible configurations for a 16-electrode lead include, but are not limited to 1-4-1-4; 6-8; 5-3-3-3-3-1 (and all rearrangements of this configuration); and 2-2-2-2-2-2-2-2. Any other suitable segmented electrode arrangements (with or without ring electrodes) can be used including, but not limited to, those disclosed in U.S. Provisional Patent Application Ser. No. 62/113,291 and U.S. Patent Applications Publication Nos. 2012/0197375 and 2015/0045864, all of which are incorporated herein by reference.

In at least some embodiments, a lead with 16 electrodes also includes 16 terminals. Many conventional control modules and connectors are designed to accept a proximal end of a lead or lead extension with an array of eight terminals. To instead have 16 terminals could extend the length of the proximal end of the lead or lead extension and a consequent increase in the size of connector or control module.

The electrodes 134 and terminals 310 (referred to herein collectively as “contacts”) can be formed using any conductive, biocompatible material. Conventionally, contacts are made of solid metals or metal alloys, such as platinum or platinum/iridium. During the manufacturing process, the contacts can be ground down using, for example, a centerless grinding process so that the outer surface of the contact is smooth and flush with the lead body.

It can be desirable, instead, to use a contact having a metal or alloy substrate with a deposited conductive coating. The conductive coating can have a larger effective surface area that a similarly shaped metal or alloy contact because the conductive coating can have a more complex microscopic surface. A larger surface area for a contact of a given size can support higher current delivery from the electrode because the larger surface area provides a lower current density. In some embodiments, the conductive coating provides a contact with higher porosity, higher charge storage capacity, lower impedance, lower resistivity, or any combination thereof when compared to a conventional solid metal/alloy contact. Grinding of the contact, however, can damage the conductive coating and, therefore, other methods for lead manufacture may be needed.

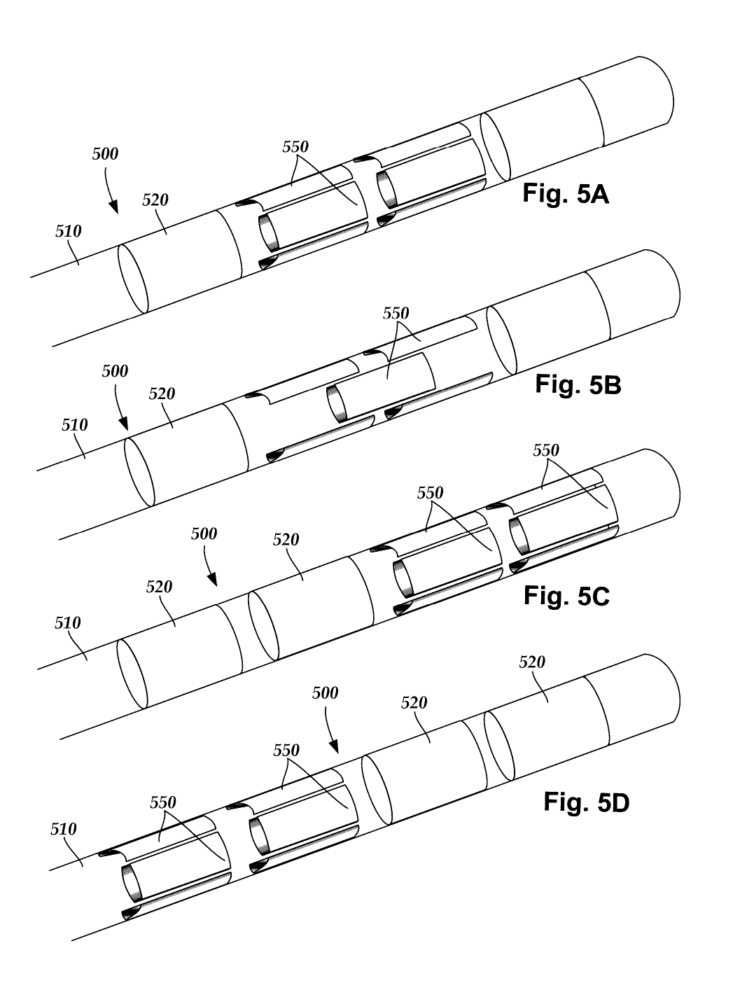

FIGS. 6-9B illustrate four different embodiments of a coated contact 660 which can be an electrode, terminal, or any other type of contact for an electrical stimulation system. The coated contact 660 includes a substrate 662 and a conductive coating 664 disposed on an outer surface of the substrate.

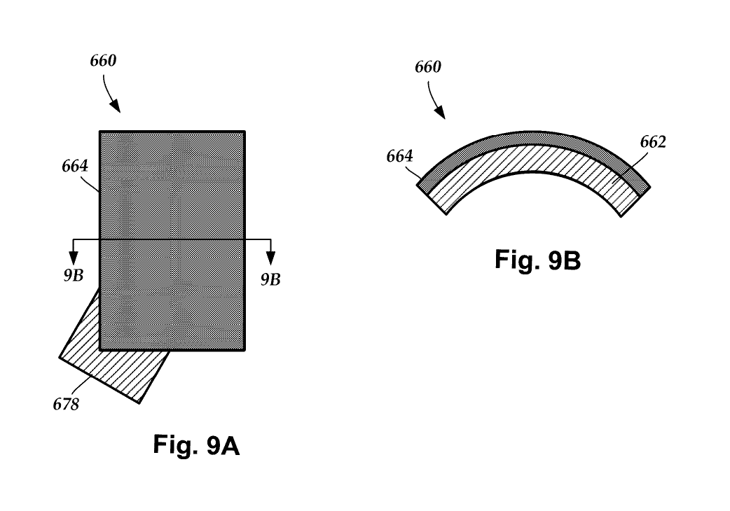

The substrate 662 can be any suitable metal or alloy including, but not limited to, platinum, platinum/iridium, titanium, and the like. The substrate 662 can be formed in any suitable shape including, as a ring as illustrated in FIGS. 6-8B, a segment as illustrate in FIGS. 9A-9B, a disk, a diamond-shaped segment, or the like. Examples of electrode shapes that can be used for the shape of the substrate 662 are illustrated in U.S. Pat. Nos. 6,295,944 and 6,391,985; and U.S. Patent Applications Publication Nos. 2011/0005069; 2010/0268298; 2011/0130817; 2011/0130818; 2011/0078900; 2011/0238129; 2011/0313500; 2012/0016378; 2012/0046710; 2012/0165911; 2012/0197375; 2012/0203316; 2012/0203320; 2012/0203321; and 2015/0045864; and U.S. Provisional Patent Application Ser. No. 62/113,291, all of which are incorporated herein by reference. The substrate 662 can also have any suitable size. The substrate 662 can be formed by any suitable method including, but not limited to, casting, cutting, or the like.

The conductive coating 664 is biocompatible and can be made of any suitable conductive material including, but not limited to, finely divided metals, such as platinum or iridium; conductive metal oxides, such as iridium oxide or tantalum oxide; conductive metal nitrides, such as titanium nitride; conductive polymers; conductive carbon coatings, such as graphite or a diamond-based material; or the like or any combination thereof. The conductive coating 664 can be applied to the substrate 662 by any suitable coating technique including, but not limited to, physical vapor deposition (PVD) techniques; chemical vapor deposition (CVD) techniques; electrodeposition; dip coating; or the like or any combination thereof. The conductive coating 664 may be a single layer or multiple layers and, if multiple layers, each layer can be the same or a different composition and thickness. In at least some embodiments, the conductive coating 664 has a thickness of at least 100 nm. In at least some embodiments, the conductive coating 664 has a thickness of in a range of 100 nm to 10 μm. FIGS. 6-9B are not to scale with respect to the relative thicknesses of the conductive coating 664 and the substrate 662. In at least some embodiments, the conductive coating 664 has a lower impedance than the substrate 662. In at least some embodiments, the conductive coating 664 has a lower resistance than the substrate 662. In at least some embodiments, the conductive coating 664 has a lower resistivity than the substrate 662. In at least some embodiments, the conductive coating 664 has a higher conductivity than the substrate 662.

Conventionally, a conductor (for example, a wire) is welded to the contact. A conductor is also attached to the coated contact 660, but welding the conductor to a surface of the substrate 662 beneath the conductive coating 664 can result in damage to the coating. FIG. 6 illustrates one embodiment in which the substrate 662 defines an opening 670 into which a portion of the conductor can be inserted and a fastening portion 672 around the opening that can be crimped or otherwise pushed against the conductor to fasten the conductor within the opening 670 and to the coated contact 660.

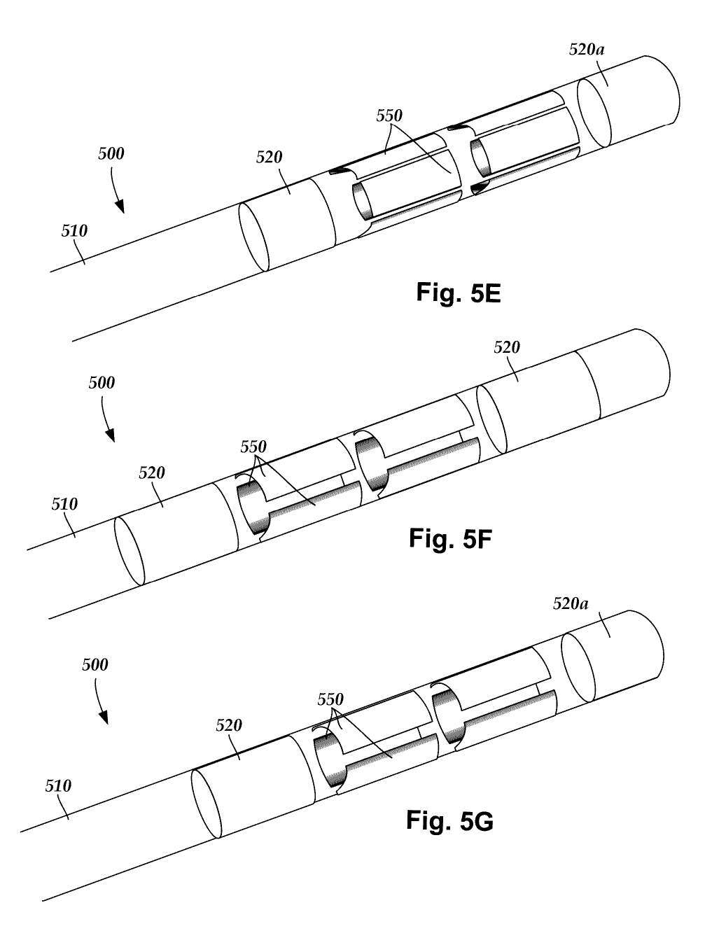

FIG. 7 illustrates another embodiment in which the substrate 662 includes two or more extensions 674 defining a gap 676 between the extensions into which a portion of the conductor can be inserted and the extensions can be crimped or otherwise pushed together to fasten the conductor within the opening gap and to the coated contact 660. The extensions 675 can be prongs, rails, or the like.

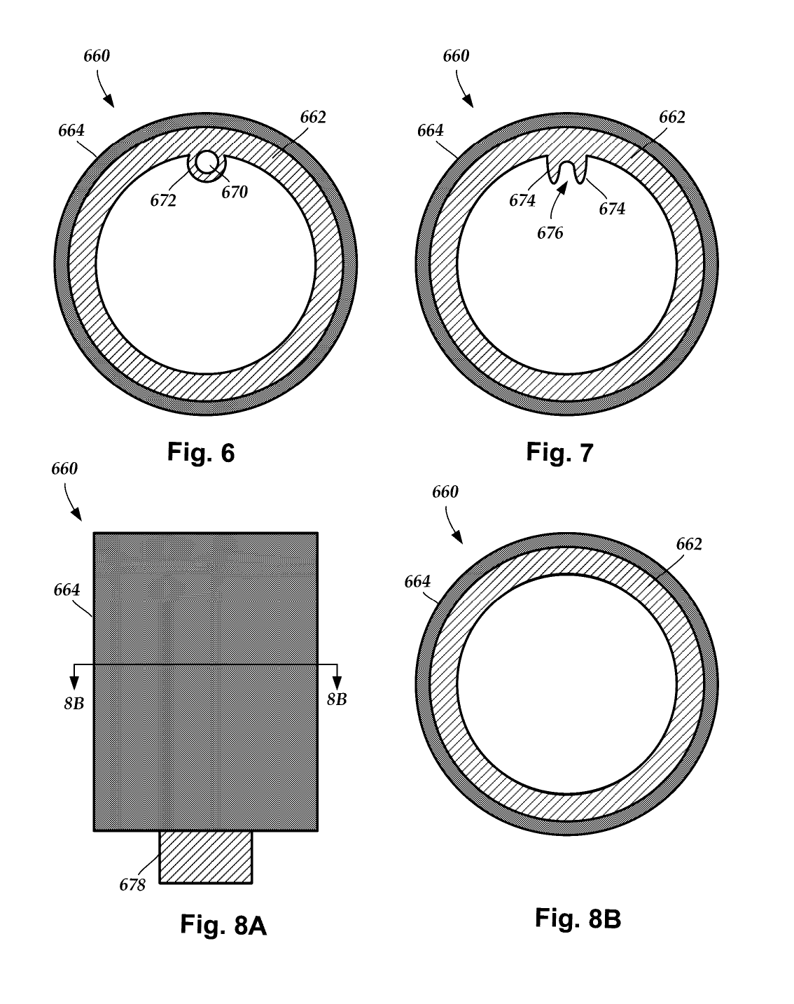

FIGS. 8A and 8B illustrate an embodiment in which the substrate 662 defines a tab 678 that is not covered by the conductive coating 664 and extends from a remainder of the substrate. The conductor can be welded or otherwise attached to the tab 678 without damaging (or with limited damage to) the conductive coating 664. In at least some embodiments, the tab 678 may be coated with the conductive coating 664, but the coating on the tab may be sacrificed during welding or other attachment process. In at least some embodiments, the tab 678 is thinner than the remainder of the substrate 662 and can be covered by the non-conductive lead body.

FIGS. 9A and 9B illustrate an embodiment of a segmented contact (such as a segmented electrode) in which the substrate 662 defines a tab 678 that is not covered by the conductive coating 664 and extends from a remainder of the substrate. The conductor can be welded or otherwise attached to the tab 678 without damaging (or with limited damage to) the conductive coating 664. In at least some embodiments, the tab 678 is thinner than the remainder of the substrate 662 and can be covered by the non-conductive lead body. In this embodiment, the tab 678 skewed relative to the remainder of the substrate 662 and conductive coating 664, as illustrated in FIG. 9A. In other embodiments, the tab 678 may be arranged similar to that illustrated in FIG. 8A.

The coated contacts described above can be used in a variety of manufacturing processes to form electrodes, terminals, or other conductive contacts of an electrical stimulation lead or lead extension. In one example of a method of manufacture of an electrical stimulation lead using the coated contacts as electrodes, the coated electrodes are placed onto a mount or fixture that holds the coated electrodes in place for attaching the conductors (for example by welding or crimping, as described above). In at least some embodiments, the mount or fixture also has lumens for feeding the conductors to the electrodes, and a center lumen to accommodate a stylet. The mount or fixture may also have a rigid component at the distal end to halt the stylet from protruding through the lead upon insertion (optional, depending upon the hardness of the distal end of the lead body). This mount or fixture can be made from a biocompatible, polymeric material such as epoxy or polyurethane which can be incorporated into the finished lead product. The mount or fixture can be a multilumen conductor guide or an end conductor guide such as those described in the previously cited references, as well as U.S. Pat. No. 8,942,810 and U.S. Patent Applications Publication Nos. 2013/0274843 and 2013/0274844, all of which are incorporated herein by reference.

This partial lead assembly (distal coated electrodes, mount or fixture, conductors, stylet back-stop, and proximal contacts) is then placed into a mold. A polymer such as silicone, epoxy, polyurethane, or the like is injected into the mold forming the lead body. In at least some embodiments, the mold is arranged so that the lead body is flush with the proximal and distal contact surfaces. In at least some embodiments, a laser may be used to remove flash, or excess polymer, that may cover the proximal and distal contacts. In some embodiments, the polymer of the lead body may be reflowed (e.g., heated again to cause the polymer to flow) to facilitate a more even lead body with the contacts. In at least some embodiments, a thin layer of a protective, water-soluble, biocompatible material such as polyethylene glycol (PEG) or similar material may be coated over the distal electrode array to protect the coated electrode material during surgical handling and implant and improve wetting of the electrode array. It will be understood that a similar process can be used to form a terminal array or other array of conductive contacts on a lead or lead extension.

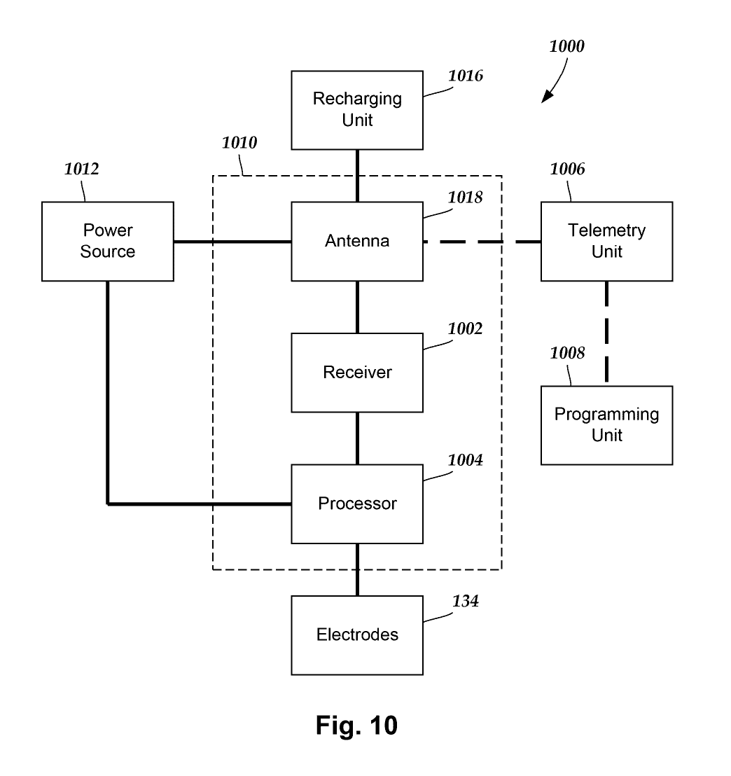

FIG. 10 is a schematic overview of one embodiment of components of an electrical stimulation system 1000 including an electronic subassembly 1010 disposed within a control module. It will be understood that the electrical stimulation system can include more, fewer, or different components and can have a variety of different configurations including those configurations disclosed in the stimulator references cited herein.

Some of the components (for example, power source 1012, antenna 1018, receiver 1002, and processor 1004) of the electrical stimulation system can be positioned on one or more circuit boards or similar carriers within a sealed housing of an implantable pulse generator, if desired. Any power source 1012 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 1018 or a secondary antenna. The external power source can be in a device that is mounted on the skin of the user or in a unit that is provided near the user on a permanent or periodic basis.

If the power source 1012 is a rechargeable battery, the battery may be recharged using the optional antenna 1018, if desired. Power can be provided to the battery for recharging by inductively coupling the battery through the antenna to a recharging unit 1016 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. A processor 1004 is generally included to control the timing and electrical characteristics of the electrical stimulation system. For example, the processor 1004 can, if desired, control one or more of the timing, frequency, strength, duration, and waveform of the pulses. In addition, the processor 1004 can select which electrodes can be used to provide stimulation, if desired. In some embodiments, the processor 1004 may select which electrode(s) are cathodes and which electrode(s) are anodes. In some embodiments, the processor 1004 may be used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 1008 that, for example, allows modification of pulse characteristics. In the illustrated embodiment, the processor 1004 is coupled to a receiver 1002 which, in turn, is coupled to the optional antenna 1018. This allows the processor 1004 to receive instructions from an external source to, for example, direct the pulse characteristics and the selection of electrodes, if desired.

In one embodiment, the antenna 1018 is capable of receiving signals (e.g., RF signals) from an external telemetry unit 1006 which is programmed by a programming unit 1008. The programming unit 1008 can be external to, or part of, the telemetry unit 1006. The telemetry unit 1006 can be a device that is worn on the skin of the user or can be carried by the user and can have a form similar to a pager, cellular phone, or remote control, if desired. As another alternative, the telemetry unit 1006 may not be worn or carried by the user but may only be available at a home station or at a clinician's office. The programming unit 1008 can be any unit that can provide information to the telemetry unit 1006 for transmission to the electrical stimulation system 1000. The programming unit 1008 can be part of the telemetry unit 1006 or can provide signals or information to the telemetry unit 1006 via a wireless or wired connection. One example of a suitable programming unit is a computer operated by the user or clinician to send signals to the telemetry unit 1006.

The signals sent to the processor 1004 via the antenna 1018 and receiver 1002 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 1000 to cease operation, to start operation, to start charging the battery, or to stop charging the battery. In other embodiments, the stimulation system does not include an antenna 1018 or receiver 1002 and the processor 1004 operates as programmed.

Optionally, the electrical stimulation system 1000 may include a transmitter (not shown) coupled to the processor 1004 and the antenna 1018 for transmitting signals back to the telemetry unit 1006 or another unit capable of receiving the signals. For example, the electrical stimulation system 1000 may transmit signals indicating whether the electrical stimulation system 1000 is operating properly or not or indicating when the battery needs to be charged or the level of charge remaining in the battery. The processor 1004 may also be capable of transmitting information about the pulse characteristics so that a user or clinician can determine or verify the characteristics.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. An electrical stimulation lead, comprising:
a lead body having a distal end portion, a proximal end portion, and a longitudinal length;
a plurality of electrodes disposed along the distal end portion of the lead body;
a plurality of terminals disposed along the proximal end portion of the lead body; and
a plurality of conductors electrically coupling the plurality of terminals to the plurality of electrodes;
wherein at least one of the electrodes or terminals is a coated contact, wherein each coated contact comprises a conductive substrate and a conductive coating disposed on the substrate.

2. The electrical stimulation lead of claim 1, wherein each of the electrodes is a one of the coated contacts.

3. The electrical stimulation lead of claim 1, wherein the conductive substrate and the conductive coating are different materials.

4. The electrical stimulation lead of claim 1, wherein the conductive coating is a conductive metal or a conductive metal oxide or a conductive metal nitride or a conductive polymer.

5. The electrical stimulation lead of claim 1, wherein the substrate of the coated contact comprises an opening receiving a portion of a one of the conductors and a fastening portion engaging the portion of the conductor to fasten the portion to the coated contact.

6. The electrical stimulation lead of claim 1, wherein the substrate of the coated contact comprises at least two extensions that define a gap receiving a portion of a one of the conductors with the at least two extensions engaging the portion of the conductor to fasten the portion to the coated contact.

7. The electrical stimulation lead of claim 1, wherein the substrate of the coated contact comprises a tab extending away from a remainder of the substrate and to which one of the conductors is attached.

8. The electrical stimulation lead of claim 7, wherein the tab is thinner than a remainder of the substrate.

9. The electrical stimulation lead of claim 1, wherein the coated contact is a ring contact.

10. The electrical stimulation lead of claim 1, wherein the coated contact extends no more than 50% around a circumference of the lead.

11. The electrical stimulation lead of claim 1, wherein the conductive coating is coated on the substrate using physical vapor deposition or chemical vapor deposition.

12. The electrical stimulation lead of claim 1, wherein the conductive coating is coated on the substrate using electrodeposition or dip coating.

13. An electrical stimulating system comprising:
the electrical stimulation lead of claim 1;
a control module coupleable to the electrical stimulation lead, the control module comprising
a housing, and
an electronic subassembly disposed in the housing; and
a connector for receiving the electrical stimulation lead, the connector having a proximal end, a distal end, and a longitudinal length, the connector comprising
a connector housing defining a port at the distal end of the connector, the port configured and arranged for receiving the proximal end of the lead body of the electrical stimulation lead, and
a plurality of connector contacts disposed in the connector housing, the plurality of connector contacts configured and arranged to couple to at least one of the plurality of terminals disposed on the proximal end of the lead body of the electrical stimulation lead.

14. The electrical stimulation system of claim 13, further comprising a lead extension coupleable to both the electrical stimulation lead and the control module.

15. A method of making the electrical stimulation lead of claim 1, the method comprising:
disposing the electrodes on a non-conductive mount, wherein at least one of the electrodes is a one of the coated contacts;
attaching the conductors to the electrodes; and
molding a lead body around the electrodes, the conductors, and the mount.

16. The method of claim 15, wherein the substrate of the coated contact comprises an opening and a fastening portion disposed around the opening, wherein attaching the conductors to the electrodes comprises inserting a portion of a one of the conductors into the opening and crimping the fastening portion to fasten the portion to the coated contact.

17. The method of claim 15, wherein the substrate of the coated contact comprises at least two extensions that define a gap, wherein attaching the conductors to the electrodes comprises inserting a portion of a one of the conductors into the gap and crimping the at least two extensions to fasten the portion to the coated contact.

18. The method of claim 15, wherein the substrate of the coated contact comprises a tab extending away from, and not radially beneath, the conductive coating, wherein attaching the conductors to the electrodes comprises welding a one of the conductors to the tab.

19. The method of claim 15, further comprising forming the coated contact by coating the substrate with the conductive coating using physical vapor deposition or chemical vapor deposition.

20. The method of claim 15, further comprising forming the coated contact by coating the substrate with the conductive coating using electrodeposition or dip coating.
